(19) United States
(12) Patent Application Publication
(10) Pub. No.: US 2010/0151042 A1
(43) Pub. Date: Jun. 17, 2010

(54) Materials and Methods for Treating Influenza Infections

(76) Inventors: Hao Yi Liang, Guangzhou (CN); Francis Chi, Hong Kong (CN); Qingfu Xu, Rochester, NY (US); Bill Piu Chan, Beijing (CN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/600,264

(22) PCT Filed: May 27, 2008
(86) PCT No.: PCT/US08/64860

§ 371 Date: Jan. 13, 2010

Related U.S. Application Data
(60) Provisional application No. 60/940,255, filed on May 25, 2007.

(51) Int. Cl. A61K 33/42; A61K 31/145; A61P 31/16; A61P 11/06; A61P 31/04; A61P 9/00; A61P 9/04; A61K 31/13; A61K 31/7056; A61K 31/7072; A61K 31/7076; A61K 31/522

(52) U.S. Cl. 424/605; 514/665; 514/662; 514/659; 514/43; 514/50; 514/46; 514/263.37

(57) ABSTRACT

The subject invention provides materials and methods for treating various health conditions, including the prevention and/or treatment of an influenza viral infection. In a preferred embodiment, a cysteamine compound and viral therapeutic are concurrently administered to a subject to treat an influenza virus infection. More preferably, a cysteamine compound is concurrently administered with a viral therapeutic to a subject to treat influenza A, influenza B, influenza C virus infections, including avian influenza virus subtypes (such as H5N1 avian influenza virus).

### BACKGROUND OF THE INVENTION

Influenza, commonly known as the “flu,” is a contagious disease that is caused by the influenza virus, classified in the orthomyxoviridae family. There are three known influenza-type viruses which affect human beings: Influenza A, B and C. Influenza A viruses have been isolated from many animal species in addition to humans, while the influenza B and C viruses have been found to infect mainly humans.

Influenza viruses are enveloped viruses containing negative single-stranded RNA's which are segmented and encapsidated. The influenza virus envelope is characterized by the presence of two surface glycoproteins: hemagglutinin and neuraminidase. The influenza A and B virions are pleomorphic and are usually 80-120 nm in diameter. The influenza C virion has many distinctive properties and is thus distinguished from the closely related A and B virions.

Influenza viruses attack the respiratory tract in humans (i.e., nose, throat, and lungs). For example, infection with influenza A or B often can cause a highly contagious, acute respiratory illness. Influenza infection usually includes the following symptoms: fever, headache, tiredness (can be extreme), dry cough, sore throat, nasal congestion, and body aches.

It is estimated that millions of people in the United States—about 10% to 20% of U.S. residents—get influenza each year. The majority of this population generally recovers in one to two weeks. In some cases, however, complications can arise from an influenza infection. Those persons at highest risk for contracting complications from the flu include: persons over 50 years of age, children aged 6 to 23 months, women more than 3 months pregnant, persons living in a long-term care facility or institution, persons with chronic heart, lung, or kidney conditions, diabetes, or weakened immune system. Pneumonia, bronchitis, encephalitis, otitis media, rhinitis, and sinusitis are only a few examples of complications that result from an influenza infection. Moreover, the flu can make chronic health problems worse. For example, people with asthma may experience asthma attacks while they have the flu, and people with chronic congestive heart failure may have worsening of this condition that is triggered by the flu.

An average of about 36,000 people per year in the United States die from influenza, and 114,000 per year have to be admitted to the hospital as a result of the infection. Thus, influenza viruses have a major impact on morbidity leading to increases in hospitalization and in visits to health care providers. For example, high rates of hospitalization are often observed for subjects over 65 years of age and also for children less than 5 years of age.

Furthermore, the spread of influenza virus through a population can result in epidemics, which have considerable economic impact. High rates of mortality were observed due to influenza infection during the influenza epidemics of 1957, 1968 and 1977 (Fields Virology, Second Edition, Volume 1, pp. 1075-1152 (1990)). Periodically, the influenza virus causes a worldwide epidemic. For example, the influenza pandemic of 1918 reportedly caused about 20 million deaths worldwide and about 500,000 deaths in the United States (Medical Microbiology, Fourth Edition, University of Texas Medical Branch at Galveston (1996)).

Influenza viruses are predominantly transmitted from person to person via respiratory droplets (also known as droplet spread) that are released as a result of coughing and/or sneezing. The influenza virus can remain suspended in the air in respiratory droplets for as long as 3 hours; but are sensitive to heat and are rapidly inactivated at temperatures above 50° C. The virus can survive for 24-48 hours on hard, non-porous surfaces (i.e., telephone receivers, computer keyboard, doorknob, kitchen countertop, toys); 8 hours on cloth, paper and tissue; and five minutes on hands (see Muir, P, “Treatment of Influenza. Essential CPE. Continuing Education from the Pharmaceutical Society of Australia,” Paragon Printers, Australasia, ACT (2002)). Typical methods of transmittal include mucous membrane contact with infected airborne respiratory droplets, person-to-person contact, contact with contaminated items (i.e., tissues soiled by infected nose and throat discharges).

Transmittal of influenza virus via respiratory droplets can occur as early as one day before a person experiences influenza-related symptoms. Adults can continue to transmit the virus to others for another three to seven days after the initial appearance of symptoms. Unlike adults, children have the ability to transmit the virus for longer than seven days. Symptoms are generally presented one to four days after the virus enters the body. In certain cases, a person can be infected with the flu virus but demonstrate no symptoms. During this time, those persons can still transmit the virus to others.

Avian influenza is an infection caused by avian (bird) influenza (flu) viruses. These influenza viruses occur naturally among birds. Avian influenza is very contagious among birds and can cause sickness and death in certain domesticated birds, including chickens, ducks, and turkeys. Because these viruses do not commonly infect humans, there is little or no immune protection against them in the human population. Of the few avian influenza viruses that have crossed the species barrier to infect humans, the strain known as H5N1 has caused the largest number of detected cases of severe disease and death in humans. Of the human cases associated with the ongoing H5N1 outbreaks in poultry and wild birds in Asia and parts of Europe, the Near East and Africa, more than half of those people reported infected with the virus have died.

Because all influenza viruses have the ability to change, scientists are concerned that H5N1 virus one day could be able to infect humans and spread easily from one person to another. If H5N1 virus were to gain the capacity to spread easily from person to person, an influenza pandemic (worldwide outbreak of disease) could begin.

Few methods are available for preventing an influenza infection, much less an avian influenza infection, and a cure has yet to be developed. Methods for preventing an influenza infection include vaccination and antiviral medications. Three antiviral drugs (amantadine, rimantadine, and oseltamivir) have been approved in the United States and are commercially available for use in preventing or treating influenza virus disease. These compounds, however, are most effective when used prophylactically, which may allow influenza viruses to develop resistance to both compounds rapidly. See U.S. Pat. Nos. 3,352,912 and 3,152,180. Other compounds reported to have activity against influenza viruses have been disclosed in U.S. Pat. Nos. 6,271,373; 5,935,957; 5,821,243; 5,684,024; 3,592,934; 3,538,160; 3,534,084; 3,496,228; and 3,483,254.

There is a great need for new therapies for the treatment of viral diseases. Whereas there has been great progress in developing a variety of therapies for the treatment of bacterial infections, there are few viable therapies for the treatment of viruses. As described above, antiviral drugs and vaccines are primary methods used in the prevention and/or treatment of influenza infections. Ganciclovir, acyclovir and foscarnet are currently utilized for the treatment of herpes virus infections. However, these therapies can have substantial side effects based on their deleterious effects on host cell DNA replication or their effect on a limited number of viral infections. In addition, as noted above, viruses are known to develop resistance to therapies, which causes a progressive decline in efficacy.

### BRIEF SUMMARY OF THE INVENTION

The subject invention provides materials and methods for treating subjects diagnosed with influenza infections as well as preventing the onset of influenza infections. In one embodiment, the invention provides methods for the treatment of influenza symptoms. In another embodiment, the subject invention provides methods for the prevention or delay in development of influenza related complications.

In a specific embodiment, the subject invention provides methods for the treatment and/or prevention of avian influenza infections and related symptoms. Specifically exemplified herein are materials and methods for the treatment of infections with the H5N1 strain of avian influenza.

A preferred embodiment of the subject invention provides a method for the treatment and/or prevention of influenza infections wherein the method involves the concurrent administration to a subject of a cysteamine compound and a second therapeutic agent useful in the treatment of viral infections. The second therapeutic agent to be used in conjunction with the cysteamine compound can be, for example, a vaccine, a neuraminidase inhibitor, or hemagglutinin inhibitor. Preferably, a cysteamine compound and a second viral therapeutic arc concurrently administered to a subject prior to, during, or after exposure to an influenza virus.

In certain embodiments of the subject invention, antitussives, mucolytics, expectorants, antipyretics, analgesics, and/or nasal decongestants can also be administered with cysteamine and the second viral therapeutic.

The subject invention is applicable to both human and animal health, especially to humans and animals infected by an influenza virus. For instance, the following, non-limiting list of viruses common to non-human animals can be treated and/or prevented in humans or animals using the present invention: swine influenza virus, equine influenza virus, equine influenza virus, avian influenza virus, feline influenza or coryza, and the like, including any mutants thereof.

Specifically exemplified herein is the concurrent administration of a cysteamine compound and a neuraminidase and/or hemagglutinin inhibitor to treat and/or prevent an influenza virus infection, including an avian influenza infection. In accordance with the subject invention, administration of a cysteamine compound concurrently with a neuraminidase or hemagluttinin inhibitor (such as oseltamivir phosphate; TAMIFLU®) to a subject prior to acquiring the influenza virus can help protect the subject from influenza infection, or at least ensure that symptoms related to influenza virus disease develop to a lesser extent than would be observed in the absence of the cysteamine compound and the second viral therapeutic, e.g., neuraminidase or hemagglutinin inhibitor.

More preferably, the present invention provides methods for the treatment and/or prevention of an avian influenza viral infection; the alleviation of avian influenza viral infection-related symptoms; as well as the prevention or delay in development of avian influenza viral infection-related complications via the concurrent administration of a cysteamine compound and a viral therapeutic.

### BRIEF DESCRIPTION OF DRAWINGS

FIG. 1 shows cysteamine as a constituent of co-enzyme A.

FIG. 2 shows a metabolic pathway of cysteamine.

### DETAILED DISCLOSURE OF THE INVENTION

The subject invention provides materials and methods for preventing and/or treating viral infections. Specifically, the subject invention provides materials and methods for preventing influenza infection; treating/ameliorating symptoms associated with influenza infections; and/or preventing/delaying the onset of complications associated with influenza infections. In preferred embodiments, the invention provides methods for preventing an avian influenza infection, treating/ameliorating symptoms associated with an avian influenza infection, as well as preventing/delaying the onset of complications associated with an avian influenza infection.

In one embodiment of the invention, a cysteamine compound is concurrently administered with a second viral therapeutic to a subject diagnosed with an influenza infection to alleviate influenza-related symptoms. According to the subject invention, a “viral therapeutic” is a treatment that treats and/or prevents an influenza virus disease. In a related embodiment, a cysteamine compound and a second viral therapeutic are administered to a subject prior to infection in order to prevent an influenza infection.

In a specific embodiment, a cysteamine compound is concurrently administered with a neuraminidase or hemagglutinin inhibitor to prevent, delay, and/or treat an influenza infection or the development of influenza-related complications in subjects who are at an increased risk of contracting those complications.

Preferably, a cysteamine compound is administered concurrently with another viral therapeutic useful in the treatment and/or prevention of the various subtypes of avian influenza virus (AIV). More preferably, a cysteamine compound of the invention is administered concurrently with another viral therapeutic useful in the treatment and/or prevention of H5N1 AIV. A dosage of at least 0.1 mg/mL of cysteamine hydrochloride, more preferably at least 1 mg/mL of cysteamine hydrochloride, and even more preferably at least 2 mg/mL of cysteamine hydrochloride, can be administered concurrently with another viral therapeutic to a subject to treat and/or prevent an AIV infection, preferably an H5N1 AIV infection.

The term “symptom(s)” as used herein, refers to signs or indications that a subject is suffering from a specific condition or disease. For example, symptoms associated with an influenza infection, as used herein, refer to signs or indications that a subject is infected with an influenza virus. Influenza-related symptoms contemplated herein include, but are not limited to, fever, headache, exhaustion/fatigue, muscular aches, sore joints, irritated watering eyes, malaise, nausea and/or vomiting, shaking, chills, chest pain, sneezing and respiratory symptoms (i.e., inflamed respiratory mucous membranes, substernal burning, nasal discharge, scratchy/sore throat, dry cough, loss of smell).

Symptoms associated with an influenza infection can start within 24 to 48 hours after infection and can begin suddenly. Chills or a chilly sensation are often the first indication of influenza. Fever is common during the first few days, and the temperature may rise to 102° F. to 103° F. In many instances, subjects feel sufficiently ill to remain in bed for days; subjects often experience aches and pains throughout the body, most pronounced in the back and legs.

As used herein, the term “complication(s)” refers to a pathological process or event occurring during a disease or condition that is not an essential part of the disease or condition; where it may result from the disease/condition or from independent causes. Accordingly, the term complication(s) refers to medical/clinical problems that arc observed in subjects diagnosed with an influenza infection. One complication of an influenza infection is that the influenza infection can make chronic health problems worse. For example, complications associated with an influenza infection include, without limitation, encephalitis, bronchitis, tracheitis, myositis rhinitis, sinusitis, asthma, bacterial infections (i.e., streptococcus aureus bacterial infection, haemophilus influenzae bacterial infection, staphylococcal pneumonia bacterial infection), cardiac complications (i.e., atrial fibrillation, myocarditis, pericarditis), Reye's syndrome, neurologic complications (i.e., confusion, convulsions, psychosis, neuritis, Guillain-Barre syndrome, coma, transverse myelitis, encephalitis, encephalomyelitis), toxic shock syndrome, myositis, myoglobinuria, and renal failure, croup, otitis media, viral infections (i.e., viral pneumonia), pulmonary fibrosis, obliterative bronchiolitis, bronchiectasis, exacerbations of asthma, exacerbations of chronic obstructive pulmonary disease, lung abscess, empyema, pulmonary aspergillosis, myositis and myoglobinaemia, heart failure, early and late fetal deaths in pregnant women, increased perinatal mortality in pregnant women, and congenital abnormalities in birth.

The terms “influenza,” “influenza virus,” or “flu,” as used herein, refer to an RNA virus of the Orthomyxoviridae family, including influenza A, influenza B, and influenza C, and mutants thereof. Influenza viruses contemplated herein include those viruses that have two antigenic glycosylated enzymes on their surface: neuraminidase and hemagglutinin. Various subtypes of influenza virus that can be treated using the materials and methods of the subject invention include, but are not limited to, the H1N1, H1N2, H2N2, H3N2, H3N8, H5N1, H5N3, H5N8, H5N9, H7N1, H7N2, H7N3, H7N4, H7N7, H9N2, and H10N7 subtypes including the following subtypes commonly known as the “Spanish Flu,” “Asian Flu,” “Hong Kong Flu,” “Avian Flu,” “Swine Flu,” “Horse Flu,” and “Dog Flu.”

The term “subject,” as used herein, describes an organism, including humans, birds, and mammals, to which treatment with the compositions according to the present invention is provided. Species that benefit from the disclosed methods of treatment include, but are not limited to, apes, chimpanzees, orangutans, humans, monkeys; and domesticated animals (i.e., pets) such as dogs, cats, horses, pigs, mice, rats, guinea pigs, hamsters, chickens, ducks, geese, and the like.

“Concurrent administration” and “concurrently administering,” as used herein, includes administering a cysteamine compound and second viral therapeutic together in a manner suitable for the treatment of a influenza infection or for the treatment of influenza infection-related symptoms/complications. As contemplated herein, concurrent administration includes providing to a subject a cysteamine compound and a second viral therapeutic as separate compounds, such as, for example, separate pharmaceutical compositions administered consecutively, simultaneously, or at different times. Preferably, if the cysteamine compound and another viral therapeutic are administered separately, they are not administered so distant in time from each other that the cysteamine compound and the viral therapeutic cannot interact. According to the subject invention, concurrent administration also encompasses providing another viral therapeutic in admixture with a cysteamine compound, such as in a phaimaceutical composition. In preferred embodiments, a cysteamine compound and second viral therapeutic are administered together at the same time.

A second viral therapeutic of the invention includes vaccinations or antiviral medications such as a neuraminidase or hemagglutinin inhibitor. Contemplated viral therapeutics for use in accordance with the subject invention include, but are not limited to, amantadine, rimantadine, ribavirin, idoxuridine, trifluridine, vidarabine, acyclovir, ganciclovir, foscarnet, zidovudine, didanosine, zalcitabine, stavudine, famciclovir, oseltamivir, zanamivir, and valaciclovir. In a preferred embodiment, the second viral therapeutic is oseltamivir phosphate.

In related embodiments, where a subject is diagnosed with an influenza infection, a cysteamine compound is concurrently administered with another viral therapeutic and other therapeutics useful in the treatment of symptoms associated with an influenza infection. For example, antitussives, mucolytics, expectorants, antipyretics, analgesics, or nasal decongestants can be concurrently administered with a cysteamine compound and second viral therapeutic to a subject diagnosed with an influenza infection.

As used herein, reference to a “cysteamine compound” includes cysteamine, the various cysteamine salts, which include pharmaceutically acceptable salts of a cysteamine compound, as well as prodrugs of cysteamine that can, for example, be readily metabolized in the body to produce cysteamine. Also included within the scope of the subject invention are analogs, derivatives, conjugates, and metabolic precursors (such as cysteine, cystamine, pantethine, and the like) as well as metabolites (such as taurine, hypotaurine, and the like) of cysteamine, which have the ability as described herein to treat and/or prevent stress and stress-related symptoms/complications by lowering cortisol levels as well as augment immune activity. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

As contemplated herein, a cysteamine compound includes pantothenic acid. Pantothenic acid is a naturally occurring vitamin that is converted in mammals to coenzyme A, a substance vital to many physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

The term “pharmaceutically acceptable salt,” as used herein, refers to any salt of a cysteamine compound that is pharmaceutically acceptable and does not greatly reduce or inhibit the activity of the cysteamine compound. Suitable examples include acid addition salts, with an organic or inorganic acid such as acetate, tartrate, trifluoroacetate, lactate, maleate, fumarate, citrate, methane, sulfonate, sulfate, phosphate, nitrate, or chloride.

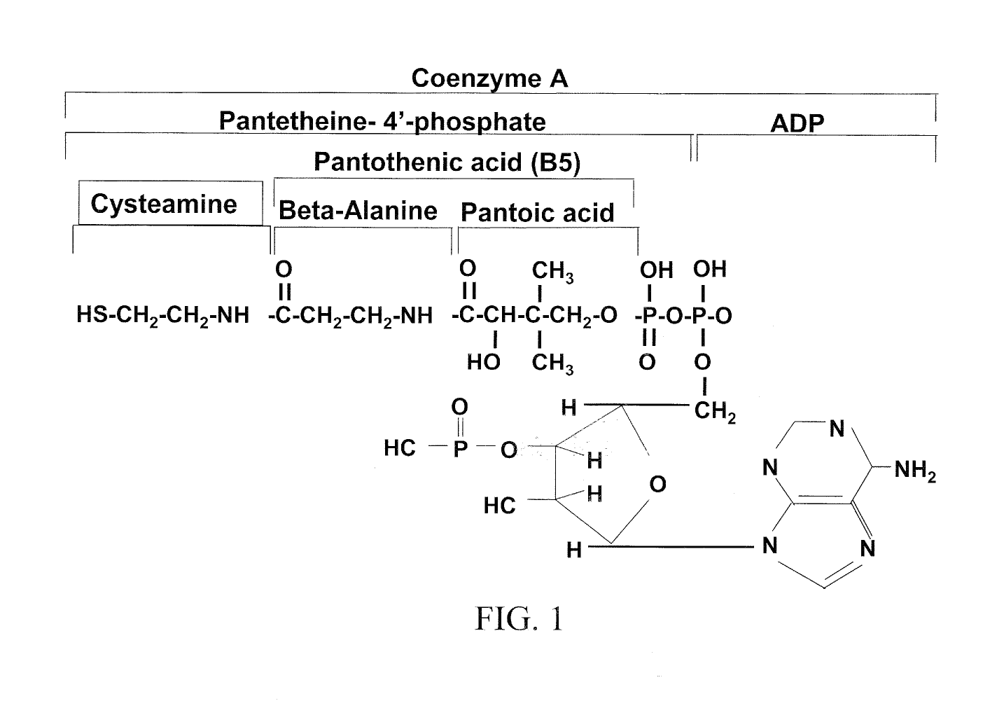

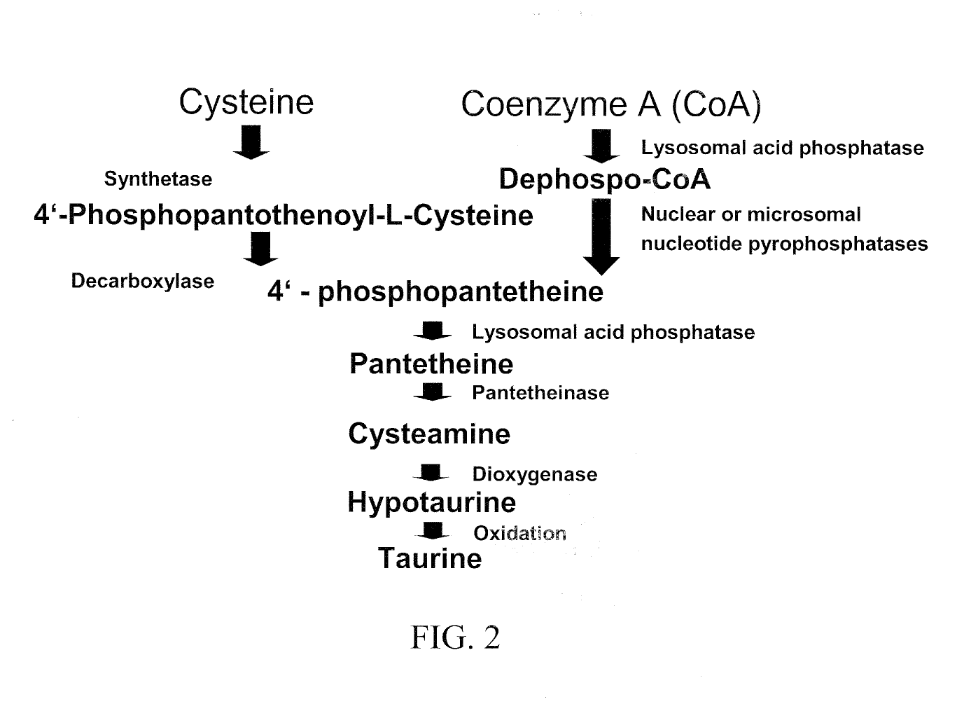

Accordingly, in one embodiment of the subject invention, the advantages of cysteamine, as set forth herein, can be achieved by promoting the endogenous production of cysteamine through natural metabolic process such as through the action of co-enzyme A or as a precursor and/or metabolite of cysteine (see FIGS. 1 and 2). This can be achieved by, for example, the administration of pantothenic acid.

The term “effective amount,” as used herein, refers to the amount necessary to elicit the desired biological response. In accordance with the subject invention, the effective amount of a cysteamine compound and another viral therapeutic is the amount necessary to treat/prevent an influenza viral infection; treat/ameliorate symptoms associated with influenza viral infections; and/or prevent/delay/ameliorate the onset of complications associated with influenza viral infections. In a preferred embodiment, the effective amount of a cysteamine compound and second viral therapeutic is the amount necessary to treat/prevent an avian influenza infection; treat/ameliorate symptoms associated with avian influenza infection; and/or prevent/delay/ameliorate the onset of complications in patients with increased risk for contracting complications associated with avian influenza infection. The amelioration in symptom and/or complication severity may be a 5%, 10%, 15%, 20%, 25% 30%, 35%, 40%, 45%, 50%, 55%, 60%, 65%, 70%, 75%, 80%, 85%, 90%, 95%, 98% or 99% decrease in severity.

The present invention is particularly applicable to non-human subject health, especially to non-human subjects infected with an influenza virus. For instance, the following, non-limited list of non-human subjects inflected with influenza virus can be treated and/or prevented using the present invention: chickens, ducks, geese, pheasants, cats, dogs, pigs, sheep, and other agriculture animals.

With regard to human subjects, the present invention is particularly applicable to the treatment and/or prevention of influenza virus infections, especially avian influenza virus infections. According to the subject invention, concurrent administration of a cysteamine compound and second viral therapeutic is useful in the treatment and/or prevention of various avian influenza strains, including viruses of subtype H1N1, H1N2, H2N2, H3N2, H3N8, H5N1, H5N2, H5N3, H5N8, H5N9, H7N1, H7N2, H7N3, H7N4, H7N7, H9N2, and H10N7. In one embodiment of the invention, cysteamine hydrochloride and another viral therapeutic are concurrently administered to subjects (either human or animal) in order to treat and/or prevent a H5N1 avian influenza virus infection.

In preferred embodiments, a cysteamine compound (such as cysteamine hydrochloride) is administered concurrently with a neuraminidase or hemagglutinin inhibitor to treat and/or prevent an avian influenza viral infection. Preferably, a cysteamine compound is concurrently administered with a neuraminidase inhibitor (such as oseltamivir phosphate) via injection or oral administration.

In one embodiment, the dosage of cysteamine and viral therapeutic administered to a subject to elicit a desired response is about 10 mg to about 3,000 mg per day for each. The desired response can include (1) prevention of influenza viral infections; preferably avian influenza infection; (2) a reduction in the severity, duration, or intensity of symptoms associated with influenza infections, preferably symptoms associated with avian influenza infection; and (3) prevention, delay, or reduction in the severity, duration, or intensity of complications related to an influenza viral infections, particularly complications related to avian influenza infections.

In one embodiment, a cysteamine compound and a second viral therapeutic are each administered at about 50 mg to 1,500 mg per day to elicit a desired response from a subject. In a related embodiment, about 200 mg to 900 mg each of cysteamine hydrochloride and another viral therapeutic are concurrently administered daily to elicit a desired response (e.g., prevent/treat the onset of an influenza infection such as avian influenza virus, influenza A, influenza B, and influenza C or any mutants thereof). Preferably, a dosage of at least 0.1 mg/mL of cysteamine hydrochloride, more preferably at least 1 mg/mL of cysteamine hydrochloride, and even more preferably at least 2 mg/mL of cysteamine hydrochloride, can be concurrently administered with another viral therapeutic to a subject to treat and/or prevent a H5N1 AIV infection.

In certain preferred embodiments, the dosage of cysteamine hydrochloride administered concurrently with a second viral therapeutic in the treatment and/or prevention of an AIV infection (including viruses of subtype H1N1, H1N2, H2N2, H3N2, H3N8, H5N1, H5N2, H5N3, H5N8, H5N9, H7N1, H7N2, H7N3, H7N4, H7N7, H9N2, and H10N7) correlates to the concentration of virus present in the subject. More preferably, the dosage of cysteamine hydrochloride administered in the treatment and/or prevention of a H5N1 AIV infection correlates to a concentration of about LD50 of virus present in the subject.

The compositions of the subject invention can include an effective amount of a cysteamine compound and second viral therapeutic. Such compositions can be used in a variety of routes of administration, including, for example, orally-administrable forms such as tablets, capsules or the like, or via parenteral, intravenous, intramuscular, transdermal, buccal, subcutaneous, suppository, or other route. These compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The cysteamine and viral therapeutic compositions of the subject invention can be formulated according to known methods for preparing pharmaceutically useful compositions. Formulations are described in a number of sources, which are well known and readily available to those skilled in the art. For example, Remington's Pharmaceutical Science (Martin E W [1995] Easton Pennsylvania, Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The formulations comprising a cysteamine compound and another viral therapeutic include those suitable for oral, rectal, nasal, topical (including buccal and sublingual), vaginal, parenteral (including subcutaneous, intramuscular, intravenous, intradermal, intrathecal and epidural) administration as well as administration to the eye. The formulations may conveniently be presented in unit dosage form and may be prepared by any of the methods well known in the art of pharmacy. Such methods include the step of bringing into association the cysteamine compound and second viral therapeutic with the carrier which constitutes one or more accessory ingredients. In general, the formulations are prepared by uniformly and intimately bringing into association the cysteamine compound and second viral therapeutic with liquid carriers or finely divided solid carriers or both, and then, if necessary, shaping the product. In certain embodiments, the cysteamine compound and second viral therapeutic are provided in a formulation for use in a skin patch.

Administration of a cysteamine compound and another viral therapeutic, in accordance with the subject invention, can be accomplished by any suitable method and technique presently or prospectively known to those skilled in the art. In a preferred embodiment, a cysteamine compound and second viral therapeutic are formulated together in a palatable and easily consumed oral formulation such as a pill, lozenge, tablet, gum, beverage, etc. The consumption is then taken at or after experiencing an influenza infection-related symptom and/or when needed to prevent an influenza infection.

Compositions of the invention preferably include as an active ingredient, an effective amount of the cysteamine compound, the second viral therapeutic, and one or more non-toxic, pharmaceutically acceptable carrier or diluent. Examples of such carriers for use in the invention include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents.

To provide for the administration of such dosages for the desired therapeutic treatment, compositions of the invention will typically comprise between about 0.1% and 95%, of the total composition including carrier or diluent. The dosage used can be varied based upon the age, weight, health, or the gender of the individual to be treated.

Following are examples that illustrate procedures for practicing the invention. These examples should not be construed as limiting. All percentages are by weight and all solvent mixture proportions are by volume unless otherwise noted.

All patents, patent applications, and publications referred to or cited herein are incorporated by reference in their entirety, including all figures and tables, to the extent they are not inconsistent with the explicit teachings of this specification. It should be understood that the examples and embodiments described herein are for illustrative purposes only and that various modifications or changes in light thereof will be suggested to persons skilled in the art and are to be included within the spirit and purview of this application.

1. A method for treating an influenza infection, wherein said method comprises diagnosing a subject with the influenza infection; and administering to the subject an effective amount of a cysteamine compound and a second viral therapeutic.

2. The method, according to claim 1, wherein the influenza viral infection is selected from the group consisting of influenza A, influenza B, and influenza C.

3. The method, according to claim 2, wherein the subject is infected with an avian influenza virus.

4. The method, according to claim 3, wherein the avian influenza virus is of a subtype selected from the group consisting of: H1N1, H1N2, H2N2, H3N2, H3N8, H5N1, H5N2, H5N3, H5N8, H5N9, H7N1, H7N2, H7N3, H7N4, H7N7, H9N2, and H10N7.

5. The method, according to claim 1, wherein the second viral therapeutic is selected from the group consisting of: amantadine, rimantadine, ribavirin, idoxuridine, trifluridine, vidarabine, acyclovir, ganciclovir, foscarnet, zidovudine, didanosine, zalcitabine, stavudine, famciclovir, oseltamivir phosphate, zanamivir, and valaciclovir.

6. The method according to claim 1, which comprises administering at least 0.1 mg of the cysteamine compound to the subject daily.

7. (canceled)

8. The method, according to claim 1, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

9. The method, according to claim 8, wherein said cysteamine salt is cysteamine hydrochloride.

10. The method, according to claim 1, wherein said cysteamine compound and said second viral therapeutic that are concurrently administered are taken orally, parenterally, intravenously, intramuscularly, transdermally, via buccal route, subcutaneously, or via suppository.

11. The method, according to claim 1, wherein said cysteamine compound and said second viral therapeutic are administered together at the same time.

12. A method for reducing the severity, intensity, or duration of complications or symptoms associated with an influenza infection, wherein said method comprises diagnosing a subject with the influenza infection; and concurrently administering to the subject an effective amount of a cysteamine compound and a second viral therapeutic.

13. The method, according to claim 12, wherein the influenza viral infection is selected from the group consisting of influenza A, influenza B, and influenza C.

14. The method, according to claim 13, wherein the subject is infected with an avian influenza virus.

15. The method, according to claim 14, wherein the avian influenza virus subtype is selected from the group consisting of: H1N1, H1N2, H2N2, H3N2, H3N8, H5N1, H5N2, H5N3, H5N8, H5N9, H7N1, H7N2, H7N3, H7N4, H7N7, H9N2, and H10N7.

16. The method, according to claim 12, wherein the complication associated with the influenza viral infection is selected from the group consisting of: encephalitis, bronchitis, tracheitis, myositis rhinitis, sinusitis, asthma, bacterial infections, cardiac complications, Reye's syndrome, neurologic complications, toxic shock syndrome, myositis, myoglobinuria, and renal failure, croup, otitis media, pulmonary fibrosis, obliterative bronchiolitis, bronchiectasis, exacerbations of asthma, exacerbations of chronic obstructive pulmonary disease, lung abscess, empyema, pulmonary aspergillosis, myositis and myoglobinaemia, heart failure, early and late fetal deaths in pregnant women, increased perinatal mortality in pregnant women, and congenital abnormalities in birth.

17. The method, according to claim 12, wherein the second viral therapeutic is selected from the group consisting of: amantadine, rimantadine, ribavirin, idoxuridine, trifluridine, vidarabine, acyclovir, ganciclovir, foscarnet, zidovudine, didanosine, zalcitabine, stavudine, famciclovir, oseltamivir phosphate, zanamivir, valaciclovir, antitussives, mucolytics, expectorants, antipyretics, analgesics, and nasal decongestants.

18-20. (canceled)

21. The method according to claim 12, wherein the cysteamine compound is cysteamine hydrochloride.

22. (canceled)

23. The method, according to claim 12, wherein said cysteamine compound and said second viral therapeutic are administered together at the same time.

24-28. (canceled)

29. The method, according to claim 12, further comprising the step of concurrently administering a therapeutic selected from the group consisting of: antitussives, mucolytics, expectorants, antipyretics, analgesics, and nasal decongestants.

30-35. (canceled)

36. A method for preventing the development of a viral infection-related complication, wherein said method comprises administering to a subject an effective amount of a cysteamine compound and a second viral therapeutic.

37-46. (canceled)
